(19) United States
(12) Patent Application Publication
(10) Pub. No.: US 2021/0015382 A1
(43) Pub. Date: Jan. 21, 2021

(54) Heart Beat Monitoring Device

(71) Applicant: Melissa Black, Chattanooga, TN (US)

(72) Inventor: Melissa Black, Chattanooga, TN (US)

(21) Appl. No.: 16/512,365

(22) Filed: Jul. 15, 2019

(51) Int. Cl. A61B 5/0402; A61B 5/0205; A61B 5/18

(52) U.S. Cl. CPC A61B 5/0402; A61M 16/0051; A61B 5/18; A61B 5/0205

(57) ABSTRACT

The present invention provides a system for vehicle security, personalization, cardiac activity and respiratory monitoring of a driver wherein active electrocardiography of a driver is mapped and in case of drivers' abnormal heart numbers or passing out under the influence of alcohol, the monitor takes the control of the vehicle and stops it precisely. The monitor further makes the automatic emergency call to pre-input contact and also provide the security to vehicle to restrict unauthorized access.

### COPYRIGHT NOTICE

A portion of the disclosure of this patent document contains material which is subject to copyright protection. The copyright owner has no objection to the xeroxographic reproduction by anyone of the patent document or the patent disclosure in exactly the form it appears in the Patent and Trademark Office patent file or records, but otherwise reserves all copyright rights whatsoever.

### FIELD OF THE INVENTION

The present invention relates to a system for vehicle security, personalization and monitoring cardiac activity of a driver. More particularly the invention relates to a use of integrated ECG (electrocardiography) device in vehicles monitoring cardiac activity of driver and taking necessary corrective action.

### DESCRIPTION OF THE RELATED ART

According to the U.S. Centers for Disease Control and Prevention, 1 in every 43 seconds Americans experience a heart attack in the average year. Most of these individuals are the first sufferers', but for more than a quarter, it's their second or more. “A heart attack occurs every 43 seconds in the United States.”

Heart attacks can occur for a variety of reasons and in a plethora of environments, often brought on by stress when underlying health factors, such as coronary artery disease, are already a problem. While driving car heart abnormal working and related conditioning can cause serious threats to the life of people siting within the vehicle and those who are driving on the road along them. To avoid such situation there are multiple types of inventions that have been brought into the market.

For instance, a U.S. Pat. No. 2,012,010,169,0A1 issued to Tata Consultancy Services Ltd discloses a system for vehicle security, personalization and cardiac activity monitoring of a driver. The present invention provides a system for vehicle security, personalization, and cardiac activity monitoring of a driver wherein electrocardiography of a driver is monitored and registered which is used for identification of a person entering in the vehicle and personalization of vehicle based on user preferences thereby act as intruder detection towards vehicle security. In addition to registration the present invention also monitors cardiac activity of driver in a continuous and real-time fashion without any intrusion to driver with the facility of generation of alert and making emergency call.

Another patent bearing U.S. Pat. No. 4,858,617A discloses a Cardiac probe enabling use of personal computer for monitoring heart activity or the like is issued to William J. Sanders. A compact and economical probe unit has projecting electrodes for sensing minute voltage variations at spaced apart locations on a persons skin or other surface. Internal circuits generate serial form digital signals indicative of the voltage variations for transmission to a personal computer where an electrocardiogram or other data presentation may be displayed. The probe includes a digital type of optical isolator through which the serial signals are transmitted, the output circuit of the isolator being energized by voltage taken from the computer and the input circuit being independently energized from a battery within the probe and thus there is no electrically conductive path between the computer and the electrodes. Among other uses, the probe enables unskilled persons to monitor their own heart activity as it is economical, safe and easily operated and makes use of a common computer for display rather than a costly electrocardiograph.

A wearable heart rate monitor bearing CN Patent 1,042,077,55A depicts an invention whom some embodiments provide a wearable fitness monitoring device including a motion sensor and a photoplethysmographic (PPG) sensor. The PPG sensor includes (i) a periodic light source, (ii) a photo detector, and (iii) circuitry determining a user's heart rate from an output of the photo detector. Some embodiments provide methods for operating a heart rate monitor of a wearable fitness monitoring device to measure one or more characteristics of a heartbeat waveform. Some embodiments provide methods for operating the wearable fitness monitoring device in a low power state when the device determines that the device is not worn by a user. Some embodiments provide methods for operating the wearable fitness monitoring device in a normal power state when the device determines that the device is worn by a user.

A vehicle-mounted multi-parameter physiological monitoring device CN1,042,877,21A depicts an invention which provides a vehicle-mounted multi-parameter physiological monitoring device. The vehicle-mounted multi-parameter physiological monitoring device is integrated on a steering wheel protective sleeve and comprises a capacitive detection module, an electrocardio acquisition module, a blood oxygen acquisition module and a circuit device. The capacitive detection module starts the vehicle-mounted multi-parameter physiological monitoring device after detecting that the palm skin of a user makes good contact with the vehicle-mounted multi-parameter physiological monitoring device. The electrocardio acquisition module acquires electrocardio signals. The blood oxygen acquisition module acquires pulse blood oxygen data. The circuit device is connected to the capacitive detection module, the electrocardio acquisition module and the blood oxygen acquisition module, receives the electrocardio signals and the pulse blood oxygen data, and gives an alarm to the user and other related personnel or organizations and establishes communication when the electrocardio signals and the pulse blood oxygen data are abnormal. The vehicle-mounted multi-parameter physiological monitoring device can guarantee that the user can be warned and treated in time when the user is physiologically abnormal in a driving process, and the driving process of the user is not influenced.

A Driver health and fatigue monitoring system and method bearing U.S. Pat. No. 8,725,311B1 is issued to American Vehicular Sciences LLC. The patent discloses a vehicle including a seat in which an occupant sits during use of the vehicle and a monitoring system for monitoring the occupant in the seat. The monitoring system includes sets of electric field antennas, each including at least one antenna, a control unit connected to the antenna sets and including selectors coupled to the antennas. The selectors are controlled by the control unit to obtain signals from one or more antennas serving as receiving antennas and one or more antennas serving as sending antennas. The control unit determines which combination of sending antenna(s) and receiving antenna(s) provides a strongest signal in an expected heartbeat range and/or expected respiration range of the occupant and then monitors this combination for changes and/or deviations from a normal range of heartbeats and/or respiration.

A Cardiac probe enabling use of personal computer for monitoring heart activity or the like bearing Canadian patent 1,326,884C is issued to ITH Inc. The patent discloses a Cardiac Probe Enabling Use of Personal Computer for Monitoring Heart Activity or the Like Abstract of the Disclosure A compact and economical probe unit has projecting electrodes for sensing minute voltage variations at spaced apart locations on a persons skin or other surface.

Internal circuits generate serial form digital signals indicative of the voltage variations for transmission to a personal computer where an electrocardiogram or other data presentation may be displayed. The probe includes a digital type of optical isolator through which the serial signals are transmitted, the output circuit of the isolator being energized by voltage taken from the computer and the input circuit being independently energized from a battery within the probe and thus there is no electrically conduct-ive path between the computer and the electrodes. Among other uses, the probe enables unskilled persons to monitor their own heart activity as it is economical, safe and easily operated and makes use of a common computer for display rather than a costly electrocardiograph.

A Health monitoring system for car seat bearing U.S. Pat. No. 6,809,643B1 issued to National Aeronautics and Space Administration (NASA) discloses a health monitoring system for use with a child car seat has sensors mounted in the seat to monitor one or more health conditions of the seat's occupant. A processor monitors the sensor's signals and generates status signals related to the monitored conditions. A transmitter wirelessly transmits the status signals to a remotely located receiver. A signaling device coupled to the receiver produces at least one sensory (e.g., visual, audible, tactile) output based on the status signals.

An automobile safety belt capable of monitoring heart rate bearing Canadian Patent CN1,042,287,55A discloses an automobile safety belt capable of monitoring heart rate and relates to the field of automobile spare parts. The safety belt comprises a belt body, a buckle body and a buckle seat. The belt body is connected with the buckle body, the buckle body is in clamping fit with the buckle seat, a sponge layer is arranged on the lower surface of the belt body, massage protrusions are arranged on the lower surface of the sponge layer, the sponge layer and the belt body are connected through a binding belt, and a Holter ECG monitor is arranged in the sponge layer and electrically connected with the buckle body. When the safety belt is bound to a user, the problem that the user feels uncomfortable due to the fact that the safety belt is hard, and the user wears the belt for a long term is solved, the heart rate change of the heart can be monitored in real time, sudden death caused by sudden heart attack in the driving process is avoided, drinking driving is avoided to certain extent, personal property loss caused by artificial reasons in the automobile running process is reduced, the service life of the Holter ECG monitor is prolonged, and the Holter ECG monitor can work all the time. The Holter ECG monitor can operate only under the condition that the safety belt is fastened, and waste caused by the fact that the Holter ECG monitor operates in an unmanned mode is avoided.

U.S. Pat. No. 6,599,243 discloses a system for advising or averting potentially dangerous driving situations based on an analysis of driver stress but it failed to disclose display of health status or ECG waveform and generation of alert with the facility of making E-call in case of emergency.

U.S. Pat. No. 5,783,997 discloses a cardiac rate measuring apparatus which measures the cardiac rate of a driver without restraining motions of the driver and U.S. Pat. No. 6,575,902 discloses a system for monitoring, recording and/or analyzing vigilance, alertness or wakefulness and/or a stressed state of an operator of equipment or machinery but both failed to disclose ECG monitoring and biometric identification of the driver with display of ECG waveform along with heart rate and also facility of making emergency call.

The current invention proposes a sophisticated system where the heart rate monitor can be attached to car seat and which can sense the abnormality in heart functioning and take the necessary required corrective actions, which can include taking control of the car in case of any emergency situation, calling the numbers in contact list, taking control of car and locking the vehicle. It also has an additional features of temperature sensing, maintaining ambient temperatures, heart rate monitoring and respiration rate monitoring.

All of the inventions listed above and many other enlisted in prior art have multiple benefits enlisted in this field. None of the previous inventions and patents, taken either singly or in combination, is seen to describe the instant invention car utility function as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary object of the invention is related to an advancement in offering heart rate monitoring device which can be attached to any vehicle.

It is further the objective of the invention to provide a heart health monitoring device which can be attached on the steering, seat belt or any other suitable position in vehicle, that senses the heart rate and takes necessary corrective actions.

It is also the objective of the invention to provide a heart monitor device which can take control of the car in case of any physical abnormality detected.

Another object of the invention is to provide real-time diagnosis of cardiac activity of vehicle driver wherein in case of an emergency an automatic emergency call or alert can be generated by the system.

It is further the object of the present invention is to provide vehicle security by preventing unauthorized access of the vehicle.

It is also the objective of the invention to provide a temperature sensing module where the infrared sensors in the steering wheel monitor the driver's facial temperature, while sensors in the steering wheels rim track changes in palms.

It is also the further objective of the present invention to provide a simplicity of its design.

It is further the objective of invention to provide a high-quality material which makes the instrument durable and long life.

It is further the object of invention to provide a drivers' heart monitoring device which is susceptible of a low cost of manufacture with regard to both materials and labor, and which accordingly is then susceptible of low prices of sale to the consuming public, thereby making such device economically available to the buying public.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way.

Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

Electronics, sensor technology and materials science techniques to monitor the development of such a patient has undergone enormous changes. In particular, many lightweight wearable devices can be used for a variety of cardiac monitoring applications. However, there remains a need to provide improved robustness wearable and intelligent device, which provide robust and efficient data collection, and in case of emergency provides immediate first aid measurements and safety measures. Other alternatives may include devices for attachment, size, flexibility, and other aspects to improve a data transmission or more aspects.

The present invention describes a medical monitoring device and system, for and/or long-term respiration data sensing and/or recording of the subject's heart rate and incase of any emergency take the necessary corrective actions.

The invention can be attached to the car steering and the monitor keeps on sensing the driver heart activity. The assembly provides heart rate and respiration monitoring. The heart rate monitoring measures changes in drivers' heart rate and piezoelectric sensor in the seatbelt counts the driver's breathing rate.

The invention as per its additional embodiments allows to provide a temperature sensing module where the infrared sensors in the steering wheel monitor the driver's facial temperature, while sensors in the steering wheels rim track changes in palms.

The invention as per its further embodiment allows to create an ambient temperature around the driver. An infrared sensor under the steering column provides a cabin temperature to compare against the driver.

Based on the heart profiling if some specific abnormality is found the invention takes the hold of the car steering and provides a steady stop to the vehicle. The monitor also makes emergency call to the already input number. The invention in its further embodiment also provides the security to the vehicle by restricting the unauthorized access.

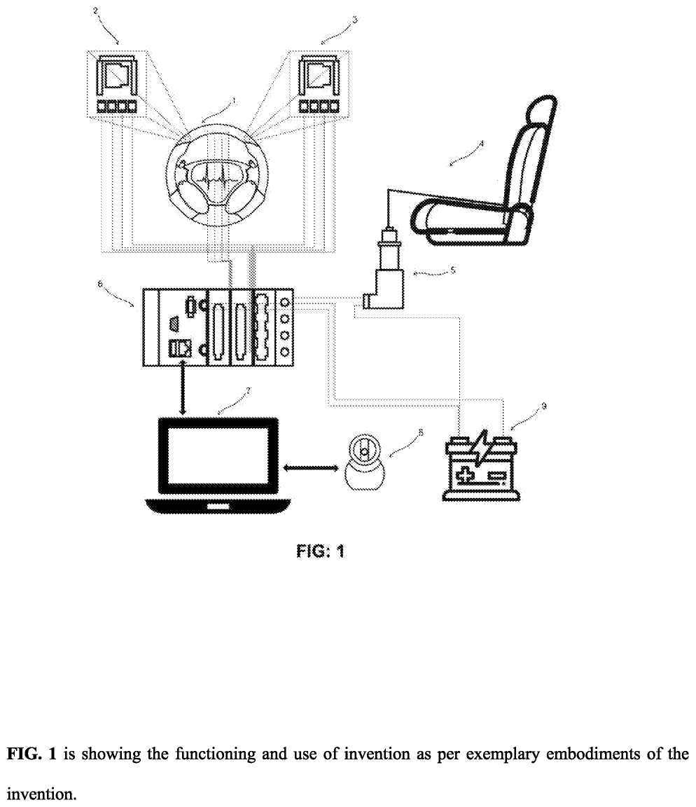

FIG. 1 is a functional block diagram of a heart beat monitoring device and method for a vehicle according to an example embodiment of the invention. The heart beat monitoring device for a vehicle (hereinafter simply referred to as “heart beat monitoring device”) 100 includes a cardiac sensor 1, a cardiac amplifier 2 and 3, a vehicle seating 4, a physical assembly of heart beat monitoring device determining portion 5, a vehicle stopping assist portion 6, a communication device 7, a display device 8, and a control apparatus 9.

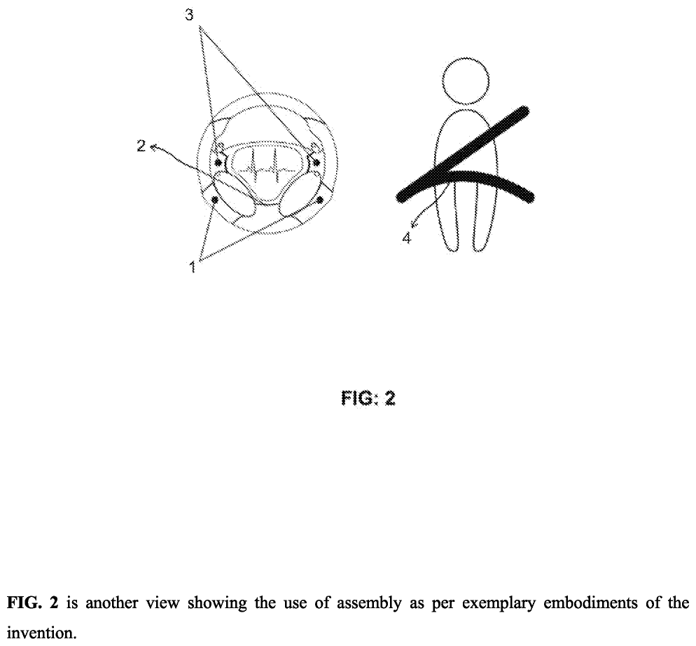

FIG. 2 is also a block diagram which is showing the placement points of the heart beat monitoring device. The device can be fixed to the seat or steering as required and convenient. The assembly is suitable for all types of vehicle.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1: The current invention aims to provide a heart monitoring device where:
the device according to claim 1, can be attached to the steering of the car;
the device according to claim 1, can be attached to the seat belt of the car, the device according to claim 1, is a mean of keeping an active profiling of the heart;
the device according to claim 1, keeps an active profiling of driver's respiration;
the device according to claim 1, provides mean of temperature sensing while rim tracks changes in palm and the device according to claim 1, provides mean of maintaining ambient temperature;

2: The heart monitoring device further provide means to alert the contacts in case the driver is detected with abnormal cardiac activity, provides means to alert the emergency pre-input contacts, stops the car and lock the car if the driver faints or passes out because of medical condition or alcohol intake.
